(19) United States
(12) Patent Application Publication
(10) Pub. No.: US 2011/0142914 A1
(43) Pub. Date: Jun. 16, 2011

(54) INHALABLE COMPOSITIONS HAVING ENHANCED BIOAVAILABILITY

(75) Inventors: Indushekhar Persaud, Homestead, FL (US); John Patrick McCook, Frisco, TX (US); Niven Rajin Narain, Miami, FL (US)

(73) Assignee: CYTOTECH LABS, LLC, Nashville, TN (US)

(21) Appl. No.: 12/746,117

(22) PCT Filed: Dec. 5, 2008
(86) PCT No.: PCT/US08/85669

§ 371 Date: Feb. 28, 2011

Related U.S. Application Data
(60) Provisional application No. 60/992,787, filed on Dec. 6, 2007.

(51) Int. Cl. A61K 9/127; A61K 31/56; A61K 31/122; A61P 29/00; A61P 33/10; A61P 9/06; A61P 31/04; A61P 31/12; A61P 7/02; A61P 25/24; A61P 3/10; A61P 25/08; A61P 31/10; A61P 19/06; A61P 9/12; A61P 33/06; A61P 25/06; A61P 35/00; A61P 15/10; A61P 37/06; A61P 33/02; A61P 25/22; A61P 25/20; A61P 9/04; A61P 25/18; A61P 7/10; A61P 25/16; A61P 1/00; A61P 17/12; A61P 9/10; A61P 3/02; A61P 5/24; A61P 21/02; A61P 25/28; A61P 3/04; A61P 19/10; A61P 31/00; A61P 39/06

(52) U.S. Cl. 424/450; 424/283.1; 514/169; 424/94.1

(57) ABSTRACT

The present disclosure provides methods and compositions suitable for delivering lipophilic bioactive agents. The compositions may be utilized to treat numerous diseases and conditions that would benefit from the application of a lipophilic bioactive agent. In embodiments the compositions may be introduced by inhalation.

### CROSS-REFERENCE TO RELATED APPLICATIONS

This application claims the benefit of and priority to U.S. Provisional Patent Application No. 60/992,787, filed Dec. 6, 2007, the entire disclosure of which is incorporated by reference herein.

### BACKGROUND

Cancer is presently one of the leading causes of death in developed nations. Although recent research has vastly increased the understanding of many of the molecular mechanisms of tumorigenesis and has provided numerous new avenues for the treatment of cancer, standard treatments for most malignancies remain gross resection, chemotherapy, and radiotherapy. While increasingly successful, each of these treatments may cause numerous undesired side effects. For example, surgery may result in pain, traumatic injury to healthy tissue, and scarring. Radiotherapy and chemotherapy may cause nausea, immune suppression, gastric ulceration and secondary tumorigenesis.

Delivery of a therapeutic agent to the respiratory tract is one avenue for the treatment of local and/or systemic diseases, including cancer. However, conventional techniques for delivery of agents to the lung may be inefficient. Attempts to develop respirable suspensions of compounds have encountered difficulty because the particles may be too large to be delivered by aerosolized droplets and fail to release the drug efficiently.

Since the vast majority of the available surface area of the lung for drug delivery is located in the deep lung, delivery to the lung may best be realized with delivery of particles to the peripheral alveoli of the deep lung. In contrast, particles deposited in the upper respiratory tract may be rapidly removed by the mucociliary escalator, subsequently transported to the throat and either swallowed or removed by coughing.

Particle formation technologies may be classified as either mechanical micronization processes or solution-based phase separation processes. Mechanical micronization methods include milling techniques such as those disclosed in U.S. Pat. No. 5,145,684. However, friction generated during these milling processes may lead to either thermal or mechanical degradation of the active agent. Spray drying, another method used to micronize drug substances, can cause difficulty with respect to capturing the particles that are formed when such particles are relatively small.

Improved methods for the treatment of diseases, including cancer, and compositions capable of delivering bioactive agents to aid in the treatment of diseases and other conditions, including by inhalation, remain desirable.

### SUMMARY

The present disclosure provides formulations and methods for the delivery of bioactive agents to the body by means of inhalation. In embodiments, the bioactive agent may be a lipophilic bioactive agent such as Coenzyme Q10 (“CoQ10”). The bioactive agent may be formed into respirable aggregates and administered to a subject by inhalation. The resulting respirable aggregates may have a mass median aerodynamic diameter of from about 1 μm to about 5 μm.

In embodiments, the bioactive agent may be in a liposomal formulation which, in turn, may be formed into respirable aggregates for administration by inhalation. Liposomal formulations which may be utilized may include a lipophilic bioactive agent prepared as a first phase, optionally in combination with a solubilizer, while a second phase is prepared containing at least one phospholipid. The two phases may be combined, thereby forming liposomes possessing the lipophilic bioactive agent. As noted above, these liposomes may then be formed into respirable aggregates and administered by inhalation.

In embodiments, a composition of the present disclosure may include at least one respirable aggregate including a liposome possessing coenzyme Q10 or a derivative thereof; and, at least one phospholipid such as lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof, wherein the respirable aggregate has a mass median aerodynamic diameter of from about 1 μm to about 5 μm.

Methods which may be utilized for forming such respirable aggregates include, but are not limited to, controlled precipitation, evaporative precipitation into aqueous solution, spray freezing into liquid, ultra-rapid freezing, high internal phase emulsion processes, combinations thereof, and the like.

In embodiments, a method of the present disclosure may include preparing a first phase including a lipophilic bioactive agent, optionally in combination with a solubilizer; preparing a second phase including at least one phospholipid; contacting the first phase with the second phase to form liposomes possessing the lipophilic bioactive agent; recovering the liposomes; and forming the liposomes into respirable aggregates having a mass median aerodynamic diameter of from about 1 μm to about 5 μm.

The aggregates may be administered in droplet form by a nebulizer or pressurized metered dose inhaler, or in a dry powder form for a dry powder inhaler.

Any medical condition which may benefit from the administration of a bioactive agent such as CoQ10 may be treated with respirable aggregates of the present disclosure.

### DETAILED DESCRIPTION

As used herein, the term “respirable aggregate” includes an aggregate of one or more particles, the aggregate having a surface area (when in dry form) of greater than about 1 m²/g. In embodiments, the surface area of the respirable aggregate may be greater than about 5 m²/g, in other embodiments greater than about 10 m²/g, and in yet other embodiments greater than about 20 m²/g. A respirable aggregate may also include smaller engineered active agent particles, each active agent particle having a particle size of less than about 1 μm. A respirable aggregate may be, for example, a dry powder or a dry powder dispersed in liquid, forming one or more droplets. The respirable aggregates of the present disclosure may also be easily wettable, as demonstrated by contact angle measurements for disks formed by pressing the respirable aggregates into tablet form. Such contact angle measurements may be less than about 50 degrees, in embodiments less than about 40 degrees, in other embodiments less than about 30 degrees, and in yet other embodiments less than about 20 degrees. Furthermore, the respirable aggregates of the present disclosure, when dry, may have a porosity of at least about 10%, in embodiments at least about 25%, in other embodiments at least about 40%, in yet other embodiments from about 60% to about 80%. The respirable aggregates of the present disclosure demonstrate a density of from about 0.1 g/mL to about 5 g/mL, in embodiments from about 0.2 g/mL to about 4 g/mL, in other embodiments from about 0.3 g/mL to about 2 g/mL, in some embodiments about 0.4 g/mL.

As used herein, the term “particle” includes a particle including an active agent, such active agents being described below in more detail. The particles may form individual units within a respirable aggregate, such that the respirable aggregate may include one or more particles possessing the active agent, dispersed throughout the respirable aggregate.

The term “fine particle fraction” as used herein includes the portion of the delivered material (i.e., a formulation that contains respirable aggregates and particles, either drops, dry powder, or the like) that is actually delivered to the lung. The fine particle fraction depends not only upon the performance of the particles and respirable aggregates, but also on the performance of the delivery device. This fine particle fraction may include respirable aggregates having a mass median aerodynamic diameter of from about 1 μm to about 5 μm. This is a suitable size for drops that are delivered by a nebulizer or pressurized metered dose inhaler (pMDI), or dry powder for a dry powder inhaler (DPI), such drops or powders including the aggregates and particles.

The terms “pharmaceutically effective amount” and “therapeutically effective amount” as used herein include a quantity or a concentration of a bioactive agent or drug that produces a desired pharmacological or therapeutic effect when administered to an animal subject, including a human. The amount of active agent or drug that includes a pharmaceutically effective amount or a therapeutically effective amount may vary according to factors such as the type of drug utilized, the potency of the particular drug, the route of administration of the formulation, the system used to administer the formulation, combinations thereof, and the like.

The terms “treatment” or “treating” herein include any treatment of a disease in a mammal, including: (i) preventing the disease, that is, causing the clinical symptoms of the disease not to develop; (ii) inhibiting the disease, that is, arresting the development of clinical symptoms; and/or (iii) relieving the disease, that is, causing regression of the clinical symptoms.

As used herein, the term “pharmaceutically acceptable carrier” includes any and all solvents, dispersion media, coatings, antibacterial and antifungal agents, stabilizing excipients, absorption enhancing or delaying agents, combinations thereof, and the like. The use of such media and agents for pharmaceutically active substances is within the purview of those skilled in the art. Supplementary active ingredients can also be incorporated into the compositions.

The term “immediate release” of an active agent from a nanoparticle as used herein describes a release profile to effect delivery of an active agent as soon as possible, that is, as soon as practically made available to an animal, whether in its active form, as a precursor, and/or as a metabolite of the agent provided.

The term “solution” as used herein includes suspensions and emulsions, as well as solutions including a bioactive agent and a solvent.

“1,3-dioxolane” is an organic solvent (commercially available, for example, from Aldrich Chemical Company, Inc.).

“BRIJ 98” is a stabilizer/solubilizer that is a polyoxyethylene 20 oleyl ether (commercially available, for example, from Sigma).

“Toluene” is an organic solvent (commercially available, for example, from Fisher Scientific).

“Dichloromethane”, sometimes referred to herein as “DCM”, is an organic solvent.

“PLURONIC F-127” is a poloxamer 407 stabilizer (commercially available, for example, from Sigma).

“Polysorbate 20” and “Polysorbate 80” are stabilizers/solubilizers (commercially available, for example, from Aldrich Chemical Company, Inc.).

“CP” means controlled precipitation, an exemplary method for making the particles and respirable aggregates of the present disclosure.

“EPAS” means evaporative precipitation into aqueous solution, an exemplary method for making the particles and respirable aggregates of the present disclosure.

“SFL” means spray freezing into liquid, an exemplary method for making the particles and respirable aggregates of the present disclosure.

“URF” means ultra-rapid freezing, an exemplary method for making the particles and respirable aggregates of the present disclosure.

“HIPE” means high internal phase emulsion, an exemplary method for making the particles and respirable aggregates of the present disclosure.

### Respirable Aggregate and Particle Preparation

The respirable aggregates of the present disclosure may be used to facilitate delivery of over 0.25 μg/g of an active agent to the deep lung. In certain embodiments delivery to the deep lung will be of at least about 1, 5, 10, 15, 20, 25 and even 30 μg/g of active agent in the lung tissue. The active agent may include a pharmaceutically acceptable carrier. The respirable aggregates may even be separated from a mixture of fractions, including those that are respirable and non-respirable.

The respirable aggregates of the present disclosure may stay in the lung (referred to herein as “residence time”) for a period of at least about 2 hours, in embodiments at least about 4 hours, in other embodiments at least about 6 hours, in other embodiments at least about 8 hours, and in yet other embodiments at least about 12 hours.

The respirable aggregates of the present disclosure may be made using any suitable method within the purview of those skilled in the art. Such methods include fast freezing methods, precipitation methods and emulsion methods.

Suitable fast freezing methods for forming respirable aggregates include those referred to herein as spray freezing into liquid (SFL), as described in U.S. Pat. No. 6,862,890, the entire disclosure of which is incorporated by reference herein, and ultra-rapid freezing (URF), as described in U.S. Patent Application Publication No. 2004/0137070, the entire disclosure of which is incorporated by reference herein.

In embodiments, a suitable SFL method may include mixing an active agent with a solution agent, and spraying the effective ingredient-solution agent mixture through an insulating nozzle located at, or below, the level of a cryogenic liquid, so that the spray generates frozen particles.

In embodiments, a suitable URF method may include contacting a solution including an active agent and at least one freezable organic solvent with a cold surface so as to freeze the solution, and removing the organic solvent.

Suitable precipitation methods for forming respirable aggregates include those referred to herein as evaporative precipitation into aqueous solution (EPAS), as described in U.S. Pat. No. 6,756,062, the entire disclosure of which is incorporated by reference herein, and controlled precipitation (CP), as described in U.S. Patent Application Publication No. 2003/0049323, the entire disclosure of which is incorporated by reference herein.

In embodiments, a suitable EPAS method may include dissolving a drug or other active agent in at least one organic solvent to form a drug/organic mixture, spraying the drug/organic mixture into an aqueous solution, while concurrently evaporating the organic solvent in the presence of the aqueous solution to form an aqueous dispersion of the drug particles.

In embodiments, a suitable CP method may include recirculating an anti-solvent through a mixing zone, dissolving a drug or other active agent in a solvent to form a solution, adding the solution to the mixing zone to form a particle slurry in the anti-solvent, and recirculating at least a portion of the particle slurry back through the mixing zone.

Suitable emulsion methods for forming respirable aggregates include those referred to herein as HIPE (high internal phase emulsions), as described in U.S. Pat. Nos. 5,539,021 and 5,688,842, the entire disclosures of each of which are incorporated by reference herein. In embodiments, a suitable HIPE method may include continuously merging into a disperser, in the presence of an emulsifying and stabilizing amount of a surfactant, a continuous phase liquid stream having a flow rate R₁, and a disperse phase liquid stream having a flow rate R₂, and mixing the merged streams with a sufficient amount of shear with R₂:R₁ sufficiently constant, to form a high internal phase ratio emulsion without phase inversion or stepwise distribution of an internal phase into an external phase.

The above methods may create particles and respirable aggregates that are crystalline or amorphous in morphology. Advantageously, none of these methods utilize mechanical milling or other similar unit operations that can cause thermal degradation of the active agent.

As noted above, in some embodiments the active agent(s) may be in solution with one or more organic solvents, and/or a combination thereof. The organic solvents may be water miscible or water immiscible. Suitable organic solvents include, but are not limited to, ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexane, heptane, pentane, 1,3-dioxolane, isopropanol, n-propanol, propionaldehyde, combinations thereof, and the like.

### Active Agents

Any bioactive agent(s) may be administered to an animal, including a human, in accordance with the present disclosure. In embodiments, suitable active agents may include lipophilic bioactive agents, sometimes referred to herein as hydrophobic bioactive agents. In embodiments the lipophilic bioactive agents may be administered as respirable aggregates formed utilizing the techniques described above. In other embodiments, the bioactive agents may be placed in liposomes, which may then be formed into respirable aggregates utilizing the techniques described above and administered to a patient. Thus, as used herein, an active agent includes both a bioactive agent and any other additives described herein, including liposomes, which may then be formed into respirable aggregates as described above.

As used herein, a lipophilic bioactive agent includes an agent that is insoluble in water. Specifically, lipophilic bioactive agents, as used herein, may have a solubility in water that is less than about 1 part of bioactive drug in about 1000 parts of water.

Suitable lipophilic bioactive agents which may be formed into respirable aggregates by themselves, or, in embodiments, included in liposomes as described above, include, but are not limited to, analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, combinations thereof, and the like.

Non-limiting examples of suitable hydrophobic active agents include, but are not limited to, acutretin, albendazole, albuterol, aminogluthemide, amiodarone, amlodipine, amphetamine, amphotericin B, atorvastatin, atovaquone, azithromycin, baclofen, beclomethsone, benezepril, benzonatate, betamethasone, bicalutanide, budesonide, bupropion, busulphan, butenafine, calcifediol, calciprotiene, calcitriol, camptothecan, candesartan, capsaicin, carbamezepine, carotenes, celecoxib, cerivistatin, cetrizine, chlorpheniramine, cholecalciferol, cilostazol, cimetidine, cinnarizine, ciprofloxacin, cisapride, clarithromycin, clemastine, clomiphene, clomipramine, clopidrogel, codeine, coenzyme Q10, cyclobenzaprine, cyclosporine, danazol, dantrolene, dexchlopheniramine, diclofenac, dicoumarol, digoxin, dihydro epiandrosterone, dihydroergotamine, dihydrotachysterol, dirithromycin, donepezil, efavirenz, eposartan, ergocalciferol, ergotamine, essential fatty acid sources, etodolac, etoposide, famotidine, fenofibrate, fentanyl, fexofenadine, finasteride, flucanazole, flurbiprofen, fluvastatin, fosphenytion, frovatriptan, furazolidone, gabapentin, gemfibrozil, glibenclamide, glipizide, glyburide, glymepride, griseofulvin, halofantrine, ibuprofen, irbesartan, irinotecan, isosorbide dinitrate, isotreinoin, itraconazole, ivermectin, ketoconazole, ketorolac, lamotrigine, lanosprazole, leflunomide, lisinopril, loperamide, loratadine, lovastatin, L-thryroxine, lutein, lycopene, medroxyprogesterone, mefepristone, mefloquine, megesterol acetate, methadone, methoxsalen, metronidazole, miconazole, midazolam, miglitol, minoxidil, mitoxantrone, montelukast, nabumetone, nalbuphine, naratiptan, nelfinavir, nifedipine, nilsolidipine, nilutanide, nitrofurantoin, nizatidine, omeprazole, oprevelkin, osteradiol, oxaprozin, paclitaxel, paricalcitol, paroxetine, pentazocine, pioglitazone, pizofetin, pravastatin, prednisolone, probucol, progesterone, pseudoephedrine, pyridostigmine, rabeprazole, raloxifene, refocoxib, repaglinide, rifabutine, rifapentine, rimexolone, ritanovir, rizatriptan, rosigiltazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terzosin, tetrahydrocannabinol, tiagabine, ticlidopine, tirofibran, tizanidine, topiramate, topotecan, toremifene, tramadol, tretinoin, troglitazone, trovafloxacin, valsartan, venlafaxine, vertoporfin, vigabatrin, vitamin A, vitamin D, vitamin E, vitamin K, zafirlukast, zileuton, zolmitriptan, zolpidem, zopiclone, combinations thereof, and the like. Salts, isomers and/or other derivatives of the above-listed bioactive agents may also be used, as well as combinations thereof.

In embodiments, coenzyme Q10 may be utilized as the bioactive agent in accordance with the present disclosure. CoQ10 is found throughout most tissues of the human body and the tissues of other mammals and is concentrated in the mitochondria. CoQ10 is very lipophilic and for the most part insoluble in water. Coenzyme Q10, sometimes referred to herein as CoQ10 or ubidecarenone, is a popular nutritional supplement and can be found in capsule form in nutritional stores, health food stores, pharmacies, and the like as a vitamin-like supplement to help protect the immune system through the antioxidant properties of ubiquinol, the reduced form of CoQ10 (ubiquinone). As used herein, Coenzyme Q10 also includes derivatives thereof, including, for example, ubiquinol. Coenzyme Q10 may be applied as respirable aggregates as described herein or also as described in International Publication No. WO 2005/069916, the entire disclosure of which is incorporated by reference herein.

In embodiments, the lipophilic bioactive agent, such as coenzyme Q10, may be combined with other bioactive agents or compounds for administration in vivo. For example, in some embodiments, combinations of bioactive agents may be utilized in accordance with the present disclosure for the treatment of cancers such as, but not limited to, lung cancer. Any bioactive agent may be combined with other bioactive agents described above, as well as additional additives and excipients described herein.

In some embodiments, the lipophilic bioactive agent, such as coenzyme Q10, may be combined with deoxyglucoses, including 2-deoxyglucose and/or 2-deoxyglucose salts, 6-deoxyglucose and/or 6-deoxyglucose salts, as a mixture or blend and administered to a patient in vivo. Suitable salts include, for example, phosphates, lactates, pyruvates, hydroxybutyrates, combinations thereof, and the like. In some embodiments, exemplary salts include phosphates such as 2-deoxyglucose phosphate, 6-deoxyglucose phosphate, combinations thereof, and the like. In other embodiments, the quinone or quinol ring of ubiquinone or ubiquinol may be substituted at the 1 position, the 4 position, or both, by the deoxyglucose or salts thereof, such as 2-deoxyglucose or 6-deoxyglucose or salts thereof, including 2-deoxyglucose phosphate or 6-deoxyglucose phosphate, with the substituted ubiquinone or ubiquinol then administered to a patient.

Similarly, in other embodiments, dihydroxy acetone may be combined with coenzyme Q10 as a mixture or blend and administered to a patient in vivo. Here also, in yet other embodiments, the quinone or quinol ring of ubiquinone or ubiquinol may be substituted at the 1 position, the 4 position, or both, with the dihydroxy acetone, with the substituted ubiquinone or ubiquinol then administered to a patient.

In yet other embodiments, compounds which may be administered with the lipophilic bioactive agent, such as coenzyme Q10, include succinates, pyruvates, citrates, fumarates, malates, malonates, lactates, glutarates, combinations thereof, and the like, with specific examples including, but not limited to, sodium succinate, potassium succinate, combinations thereof, and the like.

As noted above, in embodiments, a lipophilic bioactive agent such as coenzyme Q10, optionally in combination with other bioactive agents and/or additives, may be administered as a respirable aggregate formed utilizing the techniques described above. In other embodiments, the bioactive agents may be placed in liposomes, which may then be administered to a patient in the form of respirable aggregates formed utilizing the techniques described above. Where the bioactive agent is in a liposome, any phospholipid and/or phospholipid derivative such as a lysophospholipid may be utilized to form a liposome for encapsulating the lipophilic bioactive agent. Suitable phospholipids and/or phospholipid derivatives suitable for forming such liposomes include, but are not limited to, lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, combinations thereof, and the like.

In some embodiments, a lecithin derived from egg or soybean may be utilized as the phospholipid. Such lecithins include those commercially available as PHOSPHOLIPON® 85G, PHOSPHOLIPON® 90G, and PHOSPHOLIPON® 90H (the fully hydrogenated version of PHOSPHOLIPON® 90G) from American Lecithin Company, Oxford, Conn. Other suitable lecithins include LECINOL S-10® lecithin from Nikko Chemicals.

The above phospholipids or derivatives thereof may be utilized to form liposomes containing the bioactive agent. In embodiments, a high phosphatidylcholine content lecithin may be utilized to form a liposome. In some embodiments a high phosphatidylcholine lecithin which may be utilized includes PHOSPHOLIPON® 85G, a soy-derived lecithin containing a minimum of 85% of a linoleic acid based-phosphatidylcholine. This lecithin is easy to use and is able to produce submicron liposomes at low process temperatures (from about 20° C. to about 55° C.) without the addition of any other special additives. PHOSPHOLIPON® 85G contains, in addition to phosphatidylcholine, approximately 5-7% phosphatidic acid. The phosphatidic acid confers a negative surface charge to the resulting liposome vesicles, reduces processing time and process energy, and aids in the formation of stable liposomes.

In embodiments, it may be desirable to form a bioactive agent/liposomal concentrate, which may then be utilized to form respirable aggregates of the present disclosure. Thus, in forming a liposome, it may be desirable to combine the lipophilic bioactive agent with a material that can solubilize the lipophilic bioactive agent in a suitable media, in some embodiments water, for subsequent encapsulation in a liposome. Suitable materials which may be utilized as a solubilizer for the lipophilic bioactive agent include, for example, polyoxyalkylene dextrans, fatty acid esters of saccharose, fatty alcohol ethers of oligoglucosides (e.g., the akylpolyglucosides such as TRITON™), fatty acid esters of glycerol (e.g., glycerol mono/distearate or glycerol monolaurate), and polyoxyethylene type compounds (e.g., polyoxyethylene, polyethylene glycol, polyethylene oxide, SOLUTOL™ CREOMOPHOR™, MACROGOL™, CARBOWAX™, POLYOXYL™). Suitable solubilizers also include polyethoxylated fatty acid esters of sorbitan (e.g., Polysorbates, such as TWEEN™, SPAN™, including Polysorbate 20 and Polysorbate 80), fatty acid esters of poly(ethylene oxide) (e.g., polyoxyethylene stearates), fatty alcohol ethers of poly(ethylene oxide) (e.g., polyoxyethylated lauryl ether, polyoxyethylene 20 oleyl ether (BRIJ 98)), alkylphenol ethers of poly(ethylene oxide) (e.g., polyethoxylated octylphenol), polyoxyethylene-polyoxypropylene block copolymers (also known as poloxamers, such as “PLURONICS”, including PLURONIC F-127), and ethoxylated fats and oils (e.g., ethoxylated castor oil, or polyoxyethylated castor oil, also known as polyethylene glycol-glyceryl triricinoleate). Combinations of these solubilizers may also be utilized in embodiments. Such solubilizers and combinations are available from standard commercial sources.

In some embodiments, suitable solubilizers includes Polysorbates, e.g. those sold under the name TWEEN™. Examples of such Polysorbates include Polysorbate 80 (TWEEN™ 80), Polysorbate 20 (TWEEN™ 20), Polysorbate 60 (TWEEN™ 60), Polysorbate 65 (TWEEN™ 65), Polysorbate 85 (TWEEN™ 85), and the like, and combinations including these materials with other similar surfactants, including ARLACEL® surfactants, as long as the HLB (Hydrophile-Lipophile Balance) of the surfactant and surfactant mixture favors the formation of an O/W type emulsion system.

To assist in solubilization, it may be desirable, in embodiments, to heat the lipophilic bioactive agent and solubilizer for a suitable period of time. The temperature of heating and time of heating will depend upon the specific lipophilic bioactive agent, the intrinsic thermal stability of the bioactive agent, and solubilizer utilized. For example, in embodiments the lipophilic bioactive agent and solubilizer may be heated to a temperature of from about 40° C. to about 65° C., in embodiments from about 50° C. to about 55° C., for a period of time from about 5 minutes to about 60 minutes, in embodiments from about 15 minutes to about 30 minutes. The weight ratio of lipophilic bioactive agent to solubilizer may be about 1:1, in embodiments from about 1:1 to about 4:2, in other embodiments from about 1:2 to about 3:2.

For example, a solubilizer such as Polysorbate 80 may be capable of dissolving a lipophilic bioactive agent, in embodiments CoQ10, at high levels, with the lipophilic bioactive agent completely soluble in the solubilizer at a ratio of from about 1:2 to about 3:2, when heated to a temperature of from about 50° C. to about 55° C., a temperature which exceeds the melting point of CoQ10 (which is from about 47° C. to about 48° C.).

As noted above, the amount of solubilizer added to a lipophilic bioactive agent may depend upon the solubilizer, the lipophilic bioactive agent, and the phospholipids utilized to form the liposomes. In embodiments, a composition of the present disclosure possessing liposomes including a lipophilic bioactive agent therein may possess the solubilizer in an amount from about 0.2% to about 12% by weight, in embodiments from about 1.5% to about 6.5% by weight.

The solution of lipophilic bioactive agent and solubilizer, sometimes referred to herein as a first phase, may then be combined with a phospholipid as described above, in some embodiments lecithin. In some embodiments, it may be desirable to place the phospholipid in a dispersion, sometimes referred to herein as a second phase, to which the solution of lipophilic bioactive agent and solubilizer (i.e., the first phase) are added. Suitable solvents for forming a dispersion/second phase include, but are not limited to, water, purified water, deionized water, ethanol, isopropanol, glycols, diglycols, polyglycols, combinations thereof, and the like. Where added, the solvent may be present in an amount from about 50% by weight to about 100% by weight of the second dispersion, in embodiments from about 85% by weight to about 90% by weight of the second dispersion, with the phospholipid being present in an amount from about 5% by weight to about 20% by weight of the second dispersion, in embodiments from about 8% by weight to about 12% by weight of the second dispersion.

In some embodiments, additional components may be combined with this second phase to enhance formulation of the liposomes possessing a lipophilic bioactive agent, to improve overall rheological and processing properties of the liposomes, and to insure microbiological integrity of the resulting liposomal concentrate during storage. Such components include, without limitation, absorbents, antifoaming agents, acidifiers, alkalizers, buffers, antimicrobial agents, antioxidants (for example ascorbates, tocopherols, butylated hydroxytoluene (BHT), polyphenols, phytic acid), binders, biological additives, chelating agents (for example, disodium ethylenediamine tetraacetic acid (EDTA), tetrasodium EDTA, sodium metasilicate, and the like), denaturants, external analgesics (for example aspirin, nonsteroidal antiinflammatories and the like), steroidal antiinflammatory drugs (such as hydrocortisone and the like), preservatives (for example imidazolidinyl urea, diazolidinyl urea, phenoxyethanol, methylparaben, ethylparaben, propylparaben, and the like), reducing agents, solubilizing agents, solvents, viscosity modifiers, humectants, thickening agents, and combinations thereof. These additional components may be present in an amount from about 0.001% by weight to about 10% by weight of the second dispersion, in embodiments from about 0.1% by weight to about 1% by weight of the second dispersion.

Examples of suitable humectants include, but are not limited to, polyols and polyol derivatives, including glycerol, diglycerol, triglycerol, ethylene glycol, propylene glycol, butylene glycol, pentylene glycol (sometimes referred to herein as 1,2-pentane diol), isopreneglycol (1,4-pentane diol), 1,5-pentane diol, hexylene glycol, erythritol, 1,2,6-hexanetriol, polyethylene glycols (“PEG”) such as PEG-4, PEG-6, PEG-7, PEG-8, PEG-9, PEG-10, PEG-12, PEG-14, PEG-16, PEG-18, PEG-20, and combinations thereof, sugars and sugar derivatives (including, inter alia, fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), combinations thereof, and the like. In other embodiments, glycols such as butylene glycol, 1,2-pentane diol, glycerin, 1,5-pentane diol, combinations thereof, and the like, may be utilized as a humectant. Where utilized, any of the above humectants, including combinations thereof, may be present in amounts from about 0.1% by weight to about 20% by weight of the second dispersion, in embodiments from about 1% by weight to about 5% by weight of the second dispersion.

In some embodiments, a preservative such as phenoxyethanol and a humectant such as propylene glycol may both be added to the second phase. In embodiments, the propylene glycol may provide humectancy and assist in the preservation of the concentrate when combined with phenoxyethanol. The phenoxyethanol and propylene glycol mix should be water soluble and non-volatile. This is in contrast with the use of ethanol for preservation, which is often utilized by suppliers of liposomal dispersions. Where present, such preservatives may be present in amounts from about 0.01% by weight to about 3% by weight of the second dispersion, in embodiments from about 0.3% by weight to about 1% by weight of the second dispersion.

The dispersion containing the phospholipid, sometimes referred to herein as the second phase, and the solution containing the lipophilic bioactive agent and solubilizer, sometimes referred to herein as the first phase, may be homogenized by mixing at high shear to form a liposomal concentrate utilizing homogenizers, mixers, blenders and similar apparatus within the purview of those skilled in the art. In some embodiments, commercially available homogenizers including a Silverson L4RT Homogenizer or similar types of stator/rotor homogenizers made by Gifford-Wood, Frain, IKA and others as well as multi-stage homogenizers, colloid mills, sonolators or other types of homogenizers may be used to produce submicron liposomal dispersions of the lipophilic bioactive agent. The stator/rotor type homogenizers described above have an operational range of from about 100 rpm to about 10,000 rpm and may be supplied with a range of low shear, standard shear, and high shear head screens.

Homogenization may occur by mixing the two phases at suitable speeds of, for example, from about 4,000 rpm to about 12,000 rpm, in embodiments from about 5,000 rpm to about 10,000 rpm, in some embodiments about 7,000 rpm. The shear rate of the homogenizer may also be increased or decreased independent of the speed of the homogenizing shaft by increasing or decreasing the size of the processing screen surrounding the homogenizer head. In embodiments, liposomes may be made with both a standard emulsification screen and a high shear screen supplied for the Silverson L4RT homogenizer. Mixing may occur for a suitable period of time of less than about 90 minutes, in embodiments from about 2 minutes to about 60 minutes, in embodiments from about 5 minutes to about 45 minutes. The resulting liposomes may have a particle size of less than about 600 nm, in embodiments from about 100 nm to about 500 nm, in other embodiments from about 200 nm to about 400 nm, in some embodiments about 300 nm.

In embodiments, the two phases may be separately heated to a temperature of from about 45° C. to about 65° C., in embodiments from about 50° C. to about 55° C., and mixed with high shear homogenization at speeds and for periods of time described above to form submicron liposomes of CoQ10. Where the lipophilic bioactive agent is CoQ10, the processing temperature for the CoQ10 phase, the water/phospholipid phase, and the combined phases should not exceed about 55° C. in order to avoid oxidative degradation of the CoQ10. However, processing the mixture at a temperature from about 45° C. to about 55° C. may be desirable to obtain a desired viscosity of the concentrate of from about 5,000 cP to about 100,000 cP, in embodiments from about 15,000 cP to about 40,000 cP at from about 35° C. to about 45° C. In some embodiments, processing for extended periods, e.g., for up to about 60 minutes at the speeds noted above within this temperature range, should not adversely impact the integrity of the resulting liposomes.

The bioactive agent may be present in the resulting concentrate at a concentration of from about 15% by weight to about 30% by weight, in embodiments from about 18% by weight to about 26% by weight, in some embodiments about 22% by weight. The amount of phospholipids in the concentrate may be from about 2% to about 20% by weight, in embodiments from about 4% to about 16% by weight in the concentrate.

Once formed, the resulting liposomes may be combined with any carrier systems within the purview of those skilled in the art. As noted above, in embodiments the above liposomes may be formed into respirable aggregates utilizing the techniques described above and administered to a patient.

In embodiments, it may be desirable to include pulmonary surfactants and/or mucolytic agents in any composition including the liposomes described above in the form of respirable aggregates. Suitable pulmonary surfactants include, but are not limited to, pulmonary surfactant preparations having the function of natural pulmonary surfactant. These can include both natural and synthetic pulmonary surfactants. In embodiments, compositions which contain phospholipids and/or pulmonary surfactant proteins may be utilized.

Exemplary phospholipids which may be utilized as pulmonary surfactants according to the present disclosure include dipalmitoylphosphatidylcholine (DPPC), palmitoyloleylphosphatidylglycerol (POPG) and/or phosphatidylglycerol (PG). Other suitable phospholipids include mixtures of various phospholipids, for example, mixtures of dipalmitoylphosphatidyicholine (DPPC) and palmitoyloleylphosphatidylglycerol (POPG) at a ratio of from about 7 to about 3 to from about 3 to about 7.

Commercial products which may be utilized as pulmonary surfactant preparations include CUROSURF® (INN: PORACTANT ALFA) (Serono, Pharma GmbH, Unterschleilβheim), a natural surfactant from homogenized porcine lungs; SURVANTA® (INN: BERACTANT) (Abbott GmbH, Wiesbaden), extract of bovine lungs; ALVEOFACT® (INN: BOVACTANT) (Boehringer Ingelheim), extract of bovine lungs; EXOSURF® (INN: COLFOSCERIL PALMITATE) (Glaxo SmithKline), a synthetic phospholipid containing excipients; SURFACTEN® (INN: SURFACTANT-TA) (Mitsubishi Pharma Corporation), a pulmonary surfactant extracted from bovine lungs; INFASURF® (INN: CALFACTANT) (Forest Pharmaceuticals), a surfactant extracted from calf lungs; ALEC® (INN: PUMACTANT) (Britannia Pharmaceuticals), an artificial surfactant of DPPC and PG; and BLES® (BLES Biochemical Inc.), a bovine lipid extract surfactant.

Suitable pulmonary surfactant proteins include both proteins obtained from natural sources, such as pulmonary lavage or extraction from amniotic fluid, and proteins prepared by genetic engineering or chemical synthesis. Pulmonary surfactant proteins designated by SP-B (Surfactant Protein-B) and SP-C (Surfactant Protein-C) and their modified derivatives, including recombinant forms of the proteins, may be utilized in some embodiments.

Suitable mucolytic agents include, but are not limited to, guaifenesin, iodinated glycerol, glyceryl guaiacolate, terpin hydrate, ammonium chloride, N-acetylcysteine, bromhexine, ambroxol, iodide, their pharmaceutically acceptable salts, and combinations thereof.

In some embodiments, the amount of preservatives utilized in a composition of the present disclosure including a lipophilic bioactive agent in liposomes may also be reduced by the inclusion of additional additives including those described above. For example, the amount of preservatives may be reduced in a composition of the present disclosure by the addition of multifunctional diols including, but not limited to, 1,2-pentane diol, 1,4-pentane diol, hexylene glycol, propylene glycol, 1,3-butylene glycol, glycerol or diglycerol, combinations thereof, and the like, and by lowering the water activity, A_{w}, via the addition of humectants described above and through the addition of the soluble ingredients.

In embodiments, other soluble ingredients which may be added to compositions of the present disclosure including a lipophilic bioactive agent in liposomes may reduce the level of preservatives necessary. Such additional soluble ingredients include, but are not limited to, pH adjusting and buffering agents, tonicity adjusting agents, wetting agents and the like, for example, sodium acetate, sodium chloride, potassium chloride, calcium chloride, sorbitan monolaurate, triethanolamine oleate, and the like. Other buffers which may be added include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, citric acid, acetic acid, lactic acid, and salts of lactic acid including sodium lactate, potassium lactate, lithium lactate, calcium lactate, magnesium lactate, barium lactate, aluminum lactate, zinc lactate, sodium citrate, sodium acetate, silver lactate, copper lactate, iron lactate, manganese lactate, ammonium lactate, combinations thereof, and the like. These additives may be added to any phase described above utilized in forming the liposomes and/or respirable aggregates described above.

In embodiments, solubilization of a lipophilic bioactive agent such as CoQ10 in a material that has both lipophilic and hydrophilic properties may, in embodiments, assist in liposome formulation by forming water-dispersible CoQ10 for encapsulation by a high phosphatidylcholine lecithin, such as PHOSPHOLIPON® 85G.

In embodiments the submicron liposome concentrate formed above may be utilized to create a dosage range of respirable aggregates possessing a lipophilic bioactive agent. In embodiments, the liposome concentrate may be a CoQ10-solubilized, fluidized or emulsified within a high linoleic acid-phosphatidylcholine multilamella liposome.

### Other Excipients and Adjuvants

The above composition, in embodiments including the bioactive agent, optionally in liposomes, may be formed into respirable aggregates as described above. The bioactive agent in liposomes may be in a concentrate as described above, which may then be combined with other additives and/or excipients described above and herein to form a composition of the present disclosure suitable for the formation of respirable aggregates.

The excipients and adjuvants that may be used in the present disclosure, while potentially having some activity in their own right, for example, as antioxidants, generally include compounds that enhance the efficiency and/or efficacy of the active agents. It is also possible to have more than one excipient, adjuvant, or even active agents in a given respirable aggregate. Non-limiting examples of compounds that may be included in the respirable aggregates in accordance with the present disclosure include: surfactants, fillers, stabilizers, polymers, protease inhibitors, antioxidants, absorption enhancers, combinations thereof, and the like.

Excipients may be selected and added to the drug/organic mixture, liposomes, if present, the aqueous solution, or all of the above, either before or after the drug or bioactive age particles are formed, in order to enable the drug or bioactive age particles to be homogeneously admixed for appropriate administration. Excipients may include those items described above as suitable for formation of liposomes Other suitable excipients include polymers, absorption enhancers, solubility enhancing agents, dissolution rate enhancing agents, stability enhancing agents, bioadhesive agents, controlled release agents, flow aids and processing aids. In embodiments, suitable excipients include cellulose ethers, acrylic acid polymers, bile salts, and combinations thereof. Other suitable excipients include those described in detail in the Handbook of Pharmaceutical Excipients, published jointly by the American Pharmaceutical Association and The Pharmaceutical Society of Great Britain, the Pharmaceutical Press, 1986, relevant portions of which are incorporated by reference herein. Such excipients are commercially available and/or can be prepared by techniques within the purview of those skilled in the art.

The excipients may also be chosen alone or in combination to modify the intended function of the effective ingredients by improving flow, or bioavailability, or to control or delay the release of the active agent. Specific nonlimiting examples of excipients include: SPAN 80, TWEEN 80, BRIJ 35, BRIJ 98, PLURONICS, SUCROESTER 7, SUCROESTER 11, SUCROESTER 15, sodium lauryl sulfate, oleic acid, laureth-9, laureth-8, lauric acid, vitamin E, TPGS, GELUCIRE 50/13, GELUCIRE 53/1 0, LABRAFIL, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones, tyloxapol, cellulose derivatives, polyethoxylated castor oil derivatives, combinations thereof, and the like. In accordance with the present disclosure, the morphology of the effective ingredients can be modified, resulting in highly porous particles and respirable aggregates.

The composition of the present disclosure possessing the respirable aggregates described above, in embodiments including the liposomes and bioactive agents described above, as well as the other additives described above, may include a phospholipid in an amount of from about 0.5% to about 20% by weight of the composition, in embodiments from about 2% to about 15% by weight of the composition, with the bioactive agent present in an amount of from about 2% to about 20% by weight of the composition, in embodiments from about 5% to about 15% by weight of the composition.

### Inhalers and Nebulizers

Delivery of the respirable aggregates of the present disclosure to the lung can be achieved through any suitable delivery means, including a nebulizer, a dry powder inhaler, a pressurized metered dose inhaler, and the like. The most suitable delivery means will depend upon the active agent to be delivered to the lung, the desired effective amount for that active agent, and characteristics specific to a given patient. Details of operating such devices are within the purview of those skilled in the art.

While the instant disclosure has discussed inhalation formulations in some detail, depending on the specific conditions being treated, the lipophilic bioactive agents, described above, optionally in liposomes, may also be formulated and administered by other systemic and/or local routes. Suitable routes of administration include, but are not limited to, topical/transdermal, oral, rectal, vaginal, transmucosal, intestinal, parenteral including intramuscular, subcutaneous, intramedullary, intrathecal, direct intraventricular, intravenous, intraperitoneal, intranasal, intraocular, intratumoral, combinations thereof, and the like.

Where the compositions are by injection, the compositions may be administered in a single bolus, multiple injections, or by continuous infusion (for example, intravenously or by peritoneal dialysis). For parenteral administration, the compositions may be formulated in a sterilized pyrogen-free form. Compositions of the present disclosure can also be administered in vitro to a cell (for example, to induce apoptosis in a cancer cell in an in vitro culture) by simply adding the composition to the fluid in which the cell is contained.

### Treatments

Compositions of the present disclosure may be utilized to administer lipophilic bioactive agents for the treatment of any disease or condition which may benefit from the application of the lipophilic bioactive agent, including those disclosed in International Publication No. WO 2005/069916, the entire disclosure of which is incorporated by reference herein.

In some embodiments, compositions of the present disclosure may be utilized in the treatment of cancer. As used herein, “cancer” refers to all types of cancer or neoplasm or malignant tumors found in mammals, including, but not limited to: leukemias, lymphomas, melanomas, carcinomas and sarcomas. As used herein, the terms “cancer,” “neoplasm,” and “tumor,” are used interchangeably and in either the singular or plural form, refer to cells that have undergone a malignant transformation that makes them pathological to the host organism.

Primary cancer cells (that is, cells obtained from near the site of malignant transformation) can be readily distinguished from non-cancerous cells by well-established techniques, including histological examination. The definition of a cancer cell, as used herein, includes not only a primary cancer cell, but any cell derived from a cancer cell ancestor. This includes metastasized cancer cells, and in vitro cultures and cell lines derived from cancer cells.

When referring to a type of cancer that normally manifests as a solid tumor, a “clinically detectable” tumor is one that is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient.

Examples of cancers include cancer of the brain, breast, pancreas, cervix, colon, head and neck, kidney, lung, non-small cell lung, melanoma, mesothelioma, ovary, sarcoma, stomach, uterus and Medulloblastoma.

The term “sarcoma” generally refers to a tumor which is made up of a substance like the embryonic connective tissue and is generally composed of closely packed cells embedded in a fibrillar or homogeneous substance. Examples of sarcomas which can be treated with compositions including respirable aggregates of the present disclosure, and optionally a potentiator and/or chemotherapeutic agent include, but not limited to, a chondrosarcoma, fibrosarcoma, lymphosarcoma, melanosarcoma, myxosarcoma, osteosarcoma, adipose sarcoma, liposarcoma, alveolar soft part sarcoma, ameloblastic sarcoma, botryoid sarcoma, chloroma sarcoma, chorine carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectatic sarcoma.

The term “melanoma” includes a tumor arising from the melanocytic system of the skin and other organs. Melanomas which can be treated with compositions including respirable aggregates of the present disclosure include, but are not limited to, acral-lentiginous melanoma, amelanotic melanoma, benign juvenile I melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “carcinoma” refers to a malignant new growth made up of epithelial cells tending to infiltrate the surrounding tissues and give rise to metastases. Carcinomas which can be treated with compositions including respirable aggregates of the disclosure include, but are not limited to, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma moue, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solenoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and the like.

Additional cancers which can be treated with compositions including respirable aggregates of the present disclosure include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary; bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In addition, and as noted above, however, the compositions of the present disclosure may also be utilized to administer a lipophilic bioactive agent for the treatment of any disease or condition that may benefit from the application of a lipophilic bioactive agent.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

A concentrate was produced with CoQ10 as the lipophilic bioactive agent. About 10 kilograms (kg) of Polysorbate 80 was placed in a vacuum kettle and heated to about 50° C. to about 55° C. About 8.8 kg of CoQ10 was combined with the PHOSPHOLIPON® 85G, a vacuum was applied with the temperature maintained at about 50° C. to about 55° C., and the contents mixed for about 15 minutes. The resulting material may be referred to herein as the CoQ10 phase or the first phase. The CoQ10 was dissolved in the Polysorbate 80 with the vacuum kettle sealed, vacuum on, and temperature of the mix of Polysorbate/CoQ10 from about 50° C. to about 55° C.

In a separate kettle, about 15.8 kg of water was heated to a temperature from about 50° C. to about 55° C., and about 0.2 kg of phenoxyethanol and about 2 kg of HYDROLITE-5® pentylene glycol were added and mixed until clear and uniform. About 8 kg of PHOSPHOLIPON® 85G was then added until dispersed. The resulting material may be referred to herein as the water phase or the second phase. The water phase achieved a uniform dispersion and hydration of the lecithin and was added to the CoQ10/Polysorbate liquid as described below at 50° C. to about 55° C.

A Silverson in-line production scale homogenizer, similar to the Silverson L4RT model used for laboratory scale batches, was utilized to combine the two phases. Homogenization occurred using the Silverson standard emulsion head screen by mixing at full capacity (7000-10,000 rpm) for a total of about 5 minutes through a closed recirculating loop and under vacuum (18-20 mm Hg) at temperatures of from about 50° C. to about 55° C. with sweep agitation until the solubilized CoQ10 was completely encapsulated and uniformly dispersed to create a thick, uniform liposomal dispersion. The resulting CoQ10 concentrate possessed CoQ10 at a concentration of about 22% w/w. The Phospholipon 85 G concentration was about 8% w/w of the total composition, that is, of the combination of the two phases described above.

In separate experiments, a one kg laboratory batch of the 22% CoQ10 concentrate was made and samples were taken at 5 minute intervals during homogenization and particle size of the liposomes at that processing time was determined utilizing laser diffraction equipment (Malvern 2000) following the manufacturers directions. Details of the homogenization process and the particle sizes obtained during homogenization are set forth below in Table 1.

TABLE 1

| Process | Silver- | Avg. | Particle | Approx. peak |
|---|---|---|---|---|
| time | sonL4RT | particle | Intensity; | temp. exposure |
| (minutes) | Head Speed | diameter(nm) | % <300 nm | (degrees C.) |
| 5 | 7000 | 108 | 84.9 | 55 |
| 10 | 7000 | 162 | 57.8 | 65 |
| 15 | 7000 | 112 | 85.4 | 55 |
| 20 | 7000 | 149 | 67.0 | 62 |
| 30 | 7000 | 120 | 83.0 | 55 |
| 45 | 7000 | 107 | 85.0 | 55 |

As can be seen from Table 1, the CoQ10 concentrate formula and process described above was capable of producing liposomes with an average diameter of 107 nm and particle distribution that included 85% of all liposomes produced within a range of 59-279 nm. A short process time (5 minutes) produced a liposome dispersion of CoQ10 just as efficiently as a long process time (45 minutes). As can also be seen from the above, optimal liposome particles were obtained where the CoQ10 was not exposed to temperatures above 55° C.

The liposomal CoQ10 concentrate may be processed to form respirable aggregates in accordance with the present disclosure.

### Example 2

Preparation of particles and respirable aggregates using an SFL method. SFL powders are prepared from a homogenous organic feed solution. The feed solution contains about a 1:1 ratio of CoQ10 or the liposomal concentrate of CoQ10 produced in Example 1 above and Polysorbate 80 dissolved in acetonitrile (about 0.3% w/v total solids). The feed solution is then atomized directly into liquid nitrogen to product frozen particles. The particles are separated from the liquid nitrogen, transferred to a non-insulated container and lyophilized using a VirTis Advantage Benchtop Tray Lyophilizer (VirTis Corp., Gardiner, N.Y.) equipped with a liquid nitrogen trap to condense sublimed organic solvents. The primary drying phase is performed at about −40° C. for about 24 hours. The shelf temperature is then ramped up at a rate of about 0.9°/min to about 25° C. where the secondary drying phase is conducted for a minimum of about 12 hours. A vacuum of about 200 mTorr is maintained for the primary drying phase and increased to about 100 mTorr for the remainder of the freeze-drying cycle.

### Example 3

Nebulization of respirable aggregates produced using an SFL method. Particles from Example 2 are re-dispersed in water to form a concentration of about 20 mg/mL CoQ10 (or the liposomal concentrate of CoQ10 produced in Example 1 above). The suspension is nebulized using an Aeroneb Pro nebulizer (Aerogen Inc., Mountain View, Calif.) into an Anderson cascade impactor equipped with a 1.8 L spacer.

### Example 4

Lung residence study of respirable aggegates produced using an SFL method. A suspension prepared as in Example 3 is administered to seven-week old ICR/Swiss mice (Harlan-Sprague-Dawley, Indianapolis, Ind.), each weighing approximately 32 grams and free of disease (prior to testing). Subjects (n=14) are housed in a modified anesthesia chamber and the suspension is aerosolized into the chamber using the same device as in Example 3 to test the pulmonary efficacy of the CoQ10 formulation. Lungs are harvested from 2 separate mice and residence of the particles is determined.

### Example 5

Preparation of particles and respirable aggregates produced using an SFL method. SFL powders are prepared as in Example 2, except that a homogenous feed solution is used instead of a feed emulsion, such that the feed solution contains about a 1:0.75:0.75 ratio of CoQ10 (or the liposomal concentrate of CoQ10 produced in Example 1 above) to poloxamer 407 to Polysorbate 80 dissolved in acetonitrile (about 0.3% w/v total solids). The feed solution is then atomized directly into liquid nitrogen to product frozen particles.

### Example 6

Nebulization of respirable aggregates produced using an SFL method. The procedure described in Example 3 is performed, except that particles from Example 5 are used.

### Example 7

Lung residence study of respirable aggregates produced using an SFL method. The procedure described in Example 4 is followed, except that a suspension prepared as in Example 6 is used.

### Example 8

Preparation of particles and respirable aggregates prepared using an EPAS method. The EPAS particles and respirable aggregates are made as follows: CoQ10 or the liposomal concentrate of CoQ10 produced in example 1 above (about 15 grams) and poloxamer 407 (about 2 grams) are dissolved in dichloromethane (about 100 mL) to produce a CoQ10/organic feed solution. The CoQ10/organic feed solution is heated (to about 80° C.) and pumped (at about 1 mL/min) under pressure through an atomizing nozzle (AP=20 MPa) directly into and below the liquid level of an aqueous solution (about 100 mL) including deionized water and a particle stabilizer (about 2% (w/v) Polysorbate 80). Dichloromethane is removed during processing to leave a dispersion of particles in an aqueous solution.

### Example 9

Nebulization of respirable aggregates produced using an EPAS method. The procedure described in Example 3 is followed, except that the particles and respirable aggregates already in suspension from Example 8 are used.

### Example 10

Lung residence study of respirable aggregates produced using an EPAS method. The procedure described in Example 4 is followed, except that a suspension from Example 9 is used.

### Example 11

Preparation of dry powder from particles and respirable aggregates prepared using an EPAS method. A suspension as prepared in Example 8 is quench frozen in liquid nitrogen. This is then lyophilized as in Example 2.

### Example 12

Nebulization of respirable aggregates produced using an EPAS method. The procedure described in Example 3 is followed, except that particles from Example 11 are used.

### Example 13

Aerosolization of respirable aggregates produced using an SFL method. Particles prepared as in Example 2 are dispersed using HFA 134a into a pressurized container. The resulting sample is actuated about 5 times into an Anderson cascade impactor.

### Example 14

Preparation of particles and respirable aggregates using a URF method. A solution of CoQ10 (or the liposomal concentrate of CoQ10 produced in Example 1 above) (about 0.0798 g) with PLURONIC F-127 (about 0.0239 g) is prepared by loading the dry solids into a vial. A prepared 95/5 wt % blend of t-butanol and toluene (about 10.03 g) is loaded into the vial. The resulting slurry is heated until a solution was formed (at a temperature of from about 68° C. to about 70° C.). The resulting solution is applied to the freezing surface of the URF unit, which had been cooled to about −78° C. over a three-minute time period. The frozen solvent, drug, and excipient matrix is collected in a tray, which has been cooled with dry ice, and transferred into about a 60-mL jar, which has been cooled with dry ice. The jar containing the URF processed frozen solid is then placed on a freeze drying unit and lyophilized for approximately 17 hours at about 100 mTorr.

After lyophilization, about 0.07 grams of the URF processed solid is recovered as a dry flowable powder. The mean volume average particle sizes (with and without sonication) of the reconstituted drug particles is measured using a Coulter LS 230. The particles are amorphous.

### Example 15

Preparation of particles and respirable aggregates using a Controlled Precipitation (CP) Method. A batch controlled precipitation process is used. An aliquot of about 1.77 grams of BRIJ 98 is dissolved in about 148.33 grams of deionized water. The aqueous solution is then recirculated, using a centrifugal pump (Cole-Parmer Model 75225-10) at maximum pump speed (about 9000 rpm), through its recirculation loop and through a heat exchanger (Exergy Inc. Model 00283-01, 23 series heat exchanger) until the aqueous temperature is about 5° C. An aliquot of about 30.19 grams of a solution containing about 5 wt % CoQ10 (or the liposomal concentrate of CoQ10 produced in Example 1 above) in 1,3-dioxolane is added into the recirculating aqueous solution over about 25 seconds, which results in the controlled precipitation of a particle slurry. The particle size of the particle slurry is measured, without filtration or sonication, using a Coulter LS 230. The particle slurry is then fed to a wiped-film evaporator having a jacket temperature of about 40° C., an absolute pressure of about 8 mm Hg, and a feed rate of about 15 mL/min. The particle size of the solvent-stripped slurry is measured, without filtration or sonication, using a Coulter LS 230.

The resulting stripped slurry is freeze-dried for about 48 hours with an Edwards vacuum pump operated at maximum vacuum to isolate the drug particles. The particles are crystalline. The drug particles are reconstituted by dispersing with deionized water to a level of about 1-2 wt % solids and vortexing.

### Example 16

Particles and Respirable Aggregates prepared using an Emulsion Method. About a 2 gram aliquot of CoQ10 (or the liposomal concentrate of CoQ10 produced in Example 1 above) is dissolved in about 23 grams of methylene chloride to produce an organic solution. This solution becomes the dispersed phase. The continuous phase includes about 12.5 grams of about 2% aqueous sodium dodecyl sulfate (SDS) solution. The aqueous organic solutions are shaken together by hand to form a crude emulsion.

The emulsion is homogenized using a Fisher PowerGen 700D variable-speed motor with 20-mm diameter generator (rotor/stator) assembly for about 30 to about 60 seconds at about 20,000 rpm. A 20 gram aliquot of about 5% Methocel E3 aqueous solution is added to the emulsion along with about 16.3 grams of deionized water during homogenization. Methylene chloride is removed from the resulting mixture. The resulting suspension is freeze-dried to form a powder comprising amorphous particles.

Each isolated powder is redispersed in deionized water at about 1-2 wt % to form a slurry for particle size analysis. The particle size of the slurry is measured, without filtration or sonication, using a Coulter LS 230.

### Examples 17 and 18

Particles and Respirable Aggregates prepared using an Emulsion Method. Two other samples are prepared using the same procedure in Example 16, except sodium oleate is used instead of SDS. All isolated powders have nondetectable residual methylene chloride levels and comprise amorphous particles. Details of the aggregates of Examples 17 and 18 are summarized below in Table 2:

TABLE 2
Materials Used in Emulsion Examples 17 and 18

| | | |
|---|---|---|
| Materials used | Example 17 | Example 18 |

| | | | | |
|---|---|---|---|---|
| CoQ10 | 2 | grams | 6 | grams |
| Methylene | 23 | grams | 69 | grams |
| chloride | | | | |
| 2% aqueous | 12.5 | grams | 37.5 | grams |
| sodium oleate | | | | |
| 5% aqueous | 20 | grams | 60 | grams |
| Methocel E3 | | | | |
| Deionized | 15 | grams | 45 | grams |
| water | | | | |

### Examples 19 and 20

CoQ10 (or the liposomal concentrate of CoQ10 produced in Example 1 above) single dose pharmacokinetics in lung tissue (Example 19) and in serum (Example 20) following pulmonary administration of a CoQ10 formulation. Male Harlan-Spague-Dawley ICR mice (Hsd:ICR, Harlan Sprague Dawley, Inc., Indianapolis, Ind.) are dosed with a CoQ10 pulmonary formulation used in Example 4 using a dosing chamber.

About 20 mg/mL CoQ10 pulmonary dispersion is formed in about 4 mL of normal saline. An AERONEB PRO micro pump nebulizer (Aerogen, Inc., Mountain View, Calif.) is situated at the inlet of the chamber and nebulization of about 8 mL aliquots of the CoQ10 pulmonary dispersion is conducted over about 20 minutes for each dose. For the 24 hour pharmacokinetic study, two mice are sacrificed by carbon dioxide narcosis at each time point (0.5, 1, 2, 4, 6, 10, 24 hours), and their serum is collected and lungs are extracted and analyzed for CoQ10 content.

### Example 21

Toxicity associated with pulmonary administration of CoQ10 (or the liposomal concentrate of CoQ10 produced in Example 1 above) pulmonary formulation. Mice are dosed with about 30 mg/kg through pulmonary administration of a pulmonary CoQ10 formulation every twelve hours for up to twelve days. Observations are conducted to determine the health of mice which are administered multiple doses.

### Example 22

Multiple dose trough levels for CoQ10 (or the liposomal concentrate of CoQ10 produced in Example 1 above) delivered via pulmonary administration. Mice are dosed with about 30 mg/kg through pulmonary administration of a pulmonary CoQ10 (or the liposomal concentrate of CoQ10 produced in Example 1 above) formulation every twelve hours for up to about twelve days. About twelve hours after the last dose (trough levels) on days 3, 8 and 12, four mice are sacrificed by carbon dioxide narcosis.

Blood is collected by cardiac puncture, is allowed to clot for 20 minutes. After this time it is centrifuged and serum is collected. Surgery is performed on each mouse to extract the lung tissue which is then homogenized in 1 mL of normal saline and four 0.25 mL aliquots are analyzed for CoQ10 by reverse phase high performance liquid chromatography (HPLC).

### Example 23

Inflammatory response to the administration of CoQ10 (or the liposomal concentrate of CoQ10 produced in Example 1 above) to the lungs. Surgery is performed on sacrificed mice to expose the pleural cavity and trachea at the throat. A small incision is cut into the trachea and a cannula possessing about a 23 gauge needle with a sheath of plastic tubing (about 0.037″ outside diameter (OD) and about 0.025″ ID) is inserted through the incision to the base of the trachea and clamped to seal the opening. An aliquot (about 0.75 mL) of phosphate buffered saline is instilled through the cannula into the lungs and removed to wash the bronchial and alveolar surfaces. This process is repeated for a total of three washes. The phosphate buffered saline containing cells is placed into centrifuge vials and centrifuged at about 3000 rpm (MiniSpin Plus, Eppendorf International, Hamburg, Del.). The supernatant is removed leaving the collected cells in the pellet. The supernatant from the BAL (Bronchoalveolar Lavage) is analyzed by enzyme-linked immunosorbent assay (ELISA) for IL-12 elevation (n=2 per sample tested).

Administering CoQ10 (or the liposomal concentrate of CoQ10 produced in Example 1 above) is not connected to a rise in IL-12 levels and is not a cause of inflammation of the lung.

### Example 24

Histological analysis of mouse lungs dosed with pulmonary CoQ10 (or the liposomal concentrate of CoQ10 produced in Example 1 above). Mice are dosed via inhalation for up to about 8 days with CoQ10 (or the liposomal concentrate of CoQ10 produced in Example 1 above) formulation. Histological changes are evaluated and scored according to the Cimolai histopathologic scoring system. Lungs are harvested and placed into about 10% formaldehyde and then subjected to processing and embedding into paraffin wax. Coronal sections of the entire lung are stained and viewed by light microscopy.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A composition comprising at least one respirable aggregate comprising a liposome comprising a lipophilic bioactive agent, wherein the respirable aggregate has a mass median aerodynamic diameter of from about 1 μm to about 5 μm.

2. The composition of claim 1, wherein the liposome comprises a phospholipid selected from the group consisting of lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof.

3. The composition of claim 2, wherein the phospholipid is present in the composition in an amount from about 0.5% to about 20% by weight of the composition, and the bioactive agent is present in an amount from about 2% to about 20% by weight of the composition.

4. The composition of claim 2, wherein the phospholipid is in combination with an additional component selected from the group consisting of absorbents, antifoaming agents, acidifiers, alkalizers, buffers, antimicrobial agents, antioxidants, binders, solubilizing agents, solvents, viscosity modifiers, humectants, thickening agents, and combinations thereof.

5. The composition of claim 1, wherein the at least one lipophilic bioactive agent is selected from the group consisting of analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and combinations thereof.

6. The composition of claim 1, wherein the at least one lipophilic bioactive agent is selected from the roup consisting of acutretin, albendazole, albuterol, aminogluthemide, amiodarone, amlodipine, amphetamine, amphotericin B, atorvastatin, atovaquone, azithromycin, baclofen, beclomethsone, benezepril, benzonatate, betamethasone, bicalutanide, budesonide, bupropion, busulphan, butenafine, calcifediol, calciprotiene, calcitriol, camptothecan, candesartan, capsaicin, carbamezepine, carotenes, celecoxib, cerivistatin, cetrizine, chlorpheniramine, cholecalciferol, cilostazol, cimetidine, cinnarizine, ciprofloxacin, cisapride, clarithromycin, clemastine, clomiphene, clomipramine, clopidrogel, codeine, coenzyme Q10, cyclobenzaprine, cyclosporine, danazol, dantrolene, dexchlopheniramine, diclofenac, dicoumarol, digoxin, dihydroepiandrosterone, dihydroergotamine, dihydrotachysterol, dirithromycin, donepezil, efavirenz, eposartan, ergocalciferol, ergotamine, essential fatty acid sources, etodolac, etoposide, famotidine, fenofibrate, fentanyl, fexofenadine, finasteride, flucanazole, flurbiprofen, fluvastatin, fosphenytion, frovatriptan, furazolidone, gabapentin, gemfibrozil, glibenclamide, glipizide, glyburide, glymepride, griseofulvin, halofantrine, ibuprofen, irbesartan, irinotecan, isosorbide dinitrate, isotreinoin, itraconazole, ivermectin, ketoconazole, ketorolac, lamotrigine, lanosprazole, leflunomide, lisinopril, loperamide, loratadine, lovastatin, L-thryroxine, lutein, lycopene, medroxyprogesterone, mefepristone, mefloquine, megesterol acetate, methadone, methoxsalen, metronidazole, miconazole, midazolam, miglitol, minoxidil, mitoxantrone, montelukast, nabumetone, nalbuphine, naratiptan, nelfinavir, nifedipine, nilsolidipine, nilutanide, nitrofurantoin, nizatidine, omeprazole, oprevelkin, osteradiol, oxaprozin, paclitaxel, paricalcitol, paroxetine, pentazocine, pioglitazone, pizofetin, pravastatin, prednisolone, probucol, progesterone, pseudoephedrine, pyridostigmine, rabeprazole, raloxifene, refocoxib, repaglinide, rifabutine, rifapentine, rimexolone, ritanovir, rizatriptan, rosigiltazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terzosin, tetrahydrocannabinol, tiagabine, ticlidopine, tirofibran, tizanidine, topiramate, topotecan, toremifene, tramadol, tretinoin, troglitazone, trovafloxacin, valsartan, venlafaxine, vertoporfin, vigabatrin, vitamin A, vitamin D, vitamin E, vitamin K, zafirlukast, zileuton, zolmitriptan, zolpidem, zopiclone, and combinations thereof.

7. The composition of claim 2, wherein the phospholipid comprises lecithin, and the at least one lipophilic bioactive agent comprises coenzyme Q10.

8. The composition of claim 1, wherein the at least one lipophilic bioactive agent further comprises an additive selected from the group consisting of deoxyglucoses, deoxyglucose salts, dihydroxy acetone, succinates, pyruvates, citrates, fumarates, malates, malonates, lactates, glutarates, and combinations thereof.

9. The composition of claim 8, wherein the additive is selected from the group consisting of 2-deoxyglucose, 2-deoxyglucose phosphate, 6-deoxyglucose, 6-deoxyglucose phosphate, dihydroxy acetone, and combinations thereof.

10. The composition of claim 9, wherein the lipophilic bioactive agent comprises coenzyme Q10 substituted by the additive at the 1 position, the 4 position, or combinations thereof.

11. A method for treating cancer comprising administering the composition of claim 7 to a patient.

12. The method of claim 11, wherein the cancer comprises lung cancer.

13. A composition comprising:
at least one respirable aggregate comprising a liposome comprising coenzyme Q10 or a derivative thereof; and,
at least one phospholipid selected from the group consisting of lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof,
wherein the respirable aggregate has a mass median aerodynamic diameter of from about 1 μm to about 5 μm.

14. The composition of claim 13, wherein the phospholipid is present in the composition in an amount from about 0.5% to about 20% by weight of the composition, and the coenzyme Q10 is present in an amount from about 2% to about 20% by weight of the composition.

15. A method for treating lung cancer comprising administering the composition of claim 13 to a patient.

16. A method comprising:
preparing a first phase comprising a lipophilic bioactive agent, optionally in combination with a solubilizer;
preparing a second phase comprising at least one phospholipid;
contacting the first phase with the second phase to form liposomes possessing the lipophilic bioactive agent;
recovering the liposomes; and
forming the liposomes into respirable aggregates having a mass median aerodynamic diameter of from about 1 μm to about 5 μm.

17. The method of claim 16, wherein the at least one phospholipid is selected from the group consisting of lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof, and wherein the phospholipid is optionally in combination with an additional component selected from the group consisting of absorbents, antifoaming agents, acidifiers, alkalizers, buffers, antimicrobial agents, antioxidants, binders, solubilizing agents, solvents, viscosity modifiers, humectants, thickening agents, and combinations thereof.

18. The method of claim 16, wherein the at least one lipophilic bioactive agent is selected from the group consisting of analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and combinations thereof, and wherein the optional solubilizer is selected from the group consisting of polyoxyalkylene dextrans, fatty acid esters of saccharose, fatty alcohol ethers of oligoglucosides, fatty acid esters of glycerol, fatty acid esters of polyoxyethylenes, polyethoxylated fatty acid esters of sorbitan, fatty acid esters of poly(ethylene oxide), fatty alcohol ethers of poly(ethylene oxide), alkylphenol ethers of poly(ethylene oxide), polyoxyethylene-polyoxypropylene block copolymers, ethoxylated oils, and combinations thereof.

19. The method of claim 16, wherein the lipophilic bioactive agent comprises coenzyme Q10 and the at least one phospholipid comprises lecithin.

20. The method of claim 16, wherein forming the liposomes into respirable aggregates occurs by a process selected from the group consisting of controlled precipitation, evaporative precipitation, spray freezing into liquid, ultra-rapid freezing, high internal phase emulsion processes, and combinations thereof.

21. The method of claim 16, further comprising administering the respirable aggregates to a patient to treat lung cancer.

22. The method of claim 21, further comprising administering the respirable aggregates in droplet form.

23. The method of claim 21, wherein the respirable aggregates are administered by a method selected from the group consisting of nebulizers, pressurized metered dose inhalers, and dry powder inhalers.
